(19) United States
(12) Patent Application Publication
(10) Pub. No.: US 2023/0285767 A1
(43) Pub. Date: Sep. 14, 2023

(54) TREATMENT DEVICE USING MAGNETIC FIELD

(71) Applicant: REMED CO., LTD., Daejeon (KR)

(72) Inventors: Ghi Young KIM, Anyang-si (KR); Keun Woo KIM, Seongnam-si (KR); Chae Won HEO, Seongnam-si (KR)

(21) Appl. No.: 17/993,045

(22) Filed: Nov. 23, 2022

(30) Foreign Application Priority Data
Mar. 14, 2022 (KR) 10-2022-0031392

(51) Int. Cl. A61N 2/02

(52) U.S. Cl. CPC A61N 2/02

(57) ABSTRACT

The present invention disclosed herein relates to a treatment device using a magnetic field, which includes a coil generating a pulse magnetic field and a plurality of cooling structures.

In accordance with an embodiment of the present invention, a treatment device using a magnetic field includes: a magnetic field generating coil disposed below a close contact surface that closely contacts a portion of a body; a first blower and a second blower disposed symmetrically with respect to the magnetic field generating coil to supply a fluid toward the magnetic field generating coil; and a duct that provides a seated surface on which the magnetic field generating coil is seated and guides the fluid supplied from the first blower and the second blower toward the magnetic field generating coil, and the duct includes an opening formed by opening at least a portion of an area forming the seated surface.

### CROSS-REFERENCE TO RELATED APPLICATIONS

This U.S. non-provisional patent application claims priority under 35 U.S.C. § 119 of Korean Patent Application No. 10-2022-0031392, filed on Mar. 14, 2022, the entire contents of which are hereby incorporated by reference.

### TECHNICAL FIELD

The present invention disclosed herein relates to a treatment device using a magnetic field, and more particularly, to a treatment device using a magnetic field, which includes a coil generating a pulse magnetic field and a plurality of cooling structures.

### BACKGROUND ART

In general, a treatment device using a magnetic field induces a magnetic field by applying a pulse-type current to a coil and stimulates an affected area by inducing a current caused by the generated magnetic field into a human tissue. The treatment device using a magnetic field includes a coil generating a magnetic field by using a current applied from a main body and generates a magnetic field having a desired intensity around the coil by applying a current of several thousands amperes for a short time (50 µs to 300 µs).

Heat is inevitably generated when a current flows through the coil, and the heat increases an internal resistance of the coil and an inductance of the coil. Thus, a typical treatment device using a magnetic field essentially includes a cooling unit capable of cooling the coil.

The cooling unit uses various methods such as a water-cooling method using water, an air-cooling method that discharges heat by using a heat dissipation plate, etc., and then cools the discharged heat through a fan, etc., and an oil-cooling method that allows oil to pass around the coil and then cools the heat absorbed oil.

The typically used water-cooling method is easily maintained and has a cooling efficiency greater than that of each of the air-cooling method and the oil-cooling method. However, the water-cooling method has a low cooling efficiency because cooling water may not directly contact the coil through which a high-voltage current flows but indirectly flow around the coil for cooling the coil. Also, the water-cooling method has a limitation in that perfect insulation is required due to a risk of water leakage and external case damage.

On the other hand, the typically used air-cooling method has a limitation of a low cooling efficiency, and the oil-cooling method has a limitation of a low cooling efficiency because a high risk of leakage exists, and oil has a property of slowly heated and slowly cooled. Also, although some treatment devices adopt a compressor or radiator method as the cooling unit, the compressor or radiator method has a limitation of a lot of vibrations and noises and may not exactly control a temperature.

### Related Art Document

### Patent Document

1. Korean Patent Registration No. 10-0841596 (Title of the invention: COOLING DEVICE OF COIL FOR MAGNETIC STIMULATOR)
2. Korean Patent Registration No. 10-2020-0042301 (Title of the invention: HYBRID COLD DEVICE AND MAGNETIC STIMULATOR HAVING THE SAME)

### DISCLOSURE OF THE INVENTION

### Technical Problem

The present invention provides a treatment device using a magnetic field, which include a cooling device having a plurality of cooling structure and capable of effectively cooling heat generated from a magnetic field generating coil.

The present invention also provides a treatment device using a magnetic field, which has an intensive cooling structure capable of uniformly cooling heat generated from the coil.

The present invention also provides a treatment device using a magnetic field, which is a chair-type treatment device having a compact structure and a high cooling efficiency by designing an effective duct structure.

### Technical Solution

In accordance with an embodiment of the present invention, a treatment device using a magnetic field includes: a magnetic field generating coil disposed below a close contact surface that closely contacts a portion of a body; a first blower and a second blower disposed symmetrically with respect to the magnetic field generating coil to supply a fluid toward the magnetic field generating coil; and a duct that provides a seated surface on which the magnetic field generating coil is seated and guides the fluid supplied from the first blower and the second blower toward the magnetic field generating coil, and the duct includes an opening formed as at least a portion of an area forming the seated surface is opened.

In an embodiment, the treatment device may further include an internal electronic device disposed adjacent to the magnetic field generating coil, and the opening may guide the fluid so that the fluid supplied from the first blower and the second blower passes through the magnetic field generating coil and then is introduced toward the internal electronic device.

In an embodiment, the treatment device may further include a third blower discharging the fluid to the outside from the inside of the treatment device using a magnetic field, and the third blower may guide the fluid so that the fluid supplied from the first blower and the second blower is discharged to the outside of the treatment device using a magnetic field through the magnetic field generating coil and the internal electronic device.

In an embodiment, the internal electronic device may be disposed below the magnetic field generating coil, the opening may be defined in a region between the magnetic field generating coil and the internal electronic device, and the duct may guide the fluid so that the fluid supplied from the first blower and the second blower passes through the magnetic field generating coil in a direction parallel to a surface formed by the magnetic field generating coil, and the fluid passed through the magnetic field generating coil is supplied to the internal electronic device in a direction perpendicular to the surface formed by the magnetic field generating coil.

In an embodiment, the duct may include a hole that allows a portion of the fluid introduced to at least a portion of a region between the first blower or the second blower and the magnetic field generating coil to pass therethrough, and the hole may guide the fluid so that at least a portion of the fluid supplied from the first blower or the second blower is introduced toward the internal electronic device before being reached to the magnetic field generating coil.

### Advantageous Effects

The treatment device using a magnetic field in accordance with the present invention may include the plurality of cooling structures, i.e., the plurality of blowers for supplying the fluid to the magnetic field generating coil, to effectively cool the heat generated from the coil.

Particularly, as the velocity of the fluid passing through the magnetic field generating coil increases, the cooling efficiency at the close contact surface that closely contacts the body of the user may improve.

Also, the treatment device using a magnetic field in accordance with the present invention may prevent the limitation of over-heating of a specific portion of the coil generated while the treatment is performed by including the cooling structure capable of uniformly cooling the magnetic field generating coil.

Also, the treatment device using a magnetic field in accordance with the present invention may provide the chair-type treatment device having the compact structure and simultaneously increasing the cooling efficiency by designing the effective duct structure.

### BRIEF DESCRIPTION OF THE DRAWINGS

FIG. 1 is a perspective view illustrating a treatment device using a magnetic field in accordance with an embodiment of the present invention.

FIG. 2 is a cross-sectional view taken along line A-A and illustrating the treatment device using a magnetic field in accordance with an embodiment of the present invention.

FIG. 3 is a perspective view illustrating a treatment device using a magnetic field in accordance with another embodiment of the present invention.

FIG. 4 is a cross-sectional view taken along line B-B and illustrating the treatment device using a magnetic field in accordance with another embodiment of the present invention.

FIGS. 5a to 5e are schematic views for explaining various examples in which a plurality of blowers are disposed in treatment device using a magnetic field in accordance with various embodiments of the present invention.

FIGS. 6a to 6c are schematic views for explaining various examples in which a plurality of blowers are disposed in treatment device using a magnetic field in accordance with various embodiments of the present invention.

FIG. 7 is a schematic conceptual view illustrating a position of an inlet blower of the treatment device using a magnetic field in accordance with an embodiment of the present invention.

FIG. 8 is a schematic conceptual view illustrating a position of an outlet blower of the treatment device using a magnetic field in accordance with an embodiment of the present invention.

FIGS. 9a and 9b are cross-sectional views for comparing and explaining a cooling structure in accordance with the present invention.

FIG. 10 is an enlarged cross-sectional view illustrating region A of FIGS. 2 and 4.

FIG. 11 is a cross-sectional view taken along line A-A and illustrating a treatment device using a magnetic field in accordance with another embodiment of the present invention.

FIG. 12 is a perspective view illustrating an example in which a blower contained in the treatment device using a magnetic field in accordance with the embodiment of FIG. 11 of the present invention.

FIGS. 13 and 14 are perspective views illustrating a structure of a duct and a blower contained in the treatment device using a magnetic field in accordance with the present invention.

FIGS. 15a, 15b, 16 and 17 are schematic views for explaining a cooling fluid flow in the treatment device using a magnetic field in accordance with the embodiment of FIG. 11.

### MODE FOR CARRYING OUT THE INVENTION

Advantages and features of the present invention, and implementation methods thereof will be clarified through following embodiments described with reference to the accompanying drawings. The present invention may, however, be embodied in different forms and should not be construed as limited to the embodiments set forth herein. Rather, these embodiments are provided so that this disclosure will be thorough and complete, and will fully convey the scope of the present disclosure to those skilled in the art. Further, the present invention is only defined by scopes of claims.

It will be understood that although the terms of first and second are used herein to describe various elements, these elements should not be limited by these terms. These terms are only used to distinguish one component from another component. Accordingly, a first component that will be described below may be a second component within the technical idea of the present disclosure.

The meaning of “include,” “comprise,” “including,” or “comprising,” specifies a property, a region, a fixed number, a step, a process, an element and/or a component but does not exclude other properties, regions, fixed numbers, steps, processes, elements and/or components.

Also, for convenience of description, the dimensions of elements are exaggerated or downscaled. Therefore, it will be understood that the embodiments disclosed in this specification includes some variations without limitations to the shapes as illustrated in the figures.

Like reference numerals refer to like elements throughout.

It will also be understood that when an element is referred to as being “connected to” or “engaged with” another element, it can be directly connected to the other element, or intervening elements may also be present. It will also be understood that when an element is referred to as being ‘directly connected to’ another element, there is no intervening elements.

It will also be understood that when a layer is referred to as being “on” another layer or substrate, it can be directly on the other layer or substrate, or intervening layers may also be present. In contrast, when an element is referred to as being “directly on” another element or layer, there are no intervening elements or layers present.

Spatially relative terms, such as “below”, “beneath”, “lower”, “above”, “upper” and the like, may be used herein for ease of description to describe one element or feature’s relationship to another element(s) or feature(s) as illustrated in the figures. The spatially relative terms should be understood as terms which include different directions of configurative elements in addition to directions illustrated in the figures when using or operating the inventive concept.

Features of various embodiments of the present disclosure are partially or entirely coupled or combined with each other, and technically various interlocking and driving are enabled. Also, the embodiments may be independently performed with respect to each other, or performed in combination of each other.

Hereinafter, a treatment device using a magnetic field in accordance with the present invention will be described in detail with reference to the accompanying drawings.

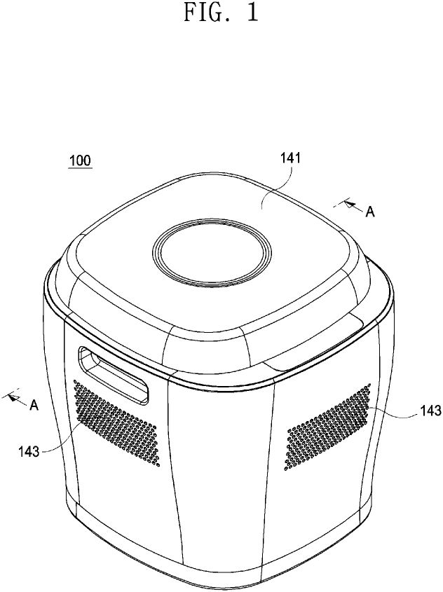

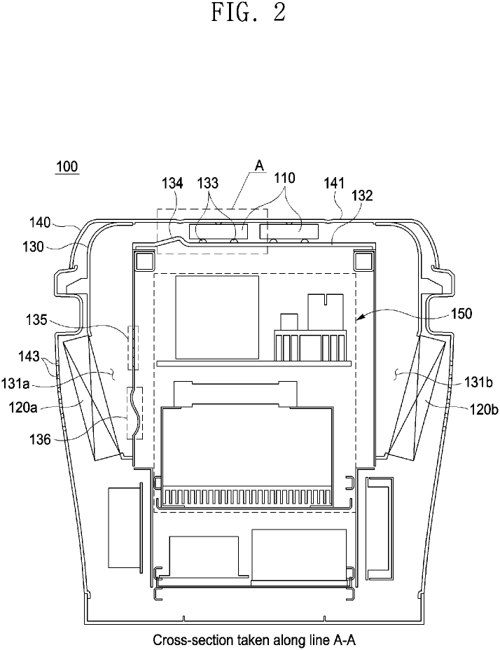

FIG. 1 is a perspective view illustrating a treatment device using a magnetic field in accordance with an embodiment of the present invention, and FIG. 2 is a cross-sectional view taken along line A-A and illustrating the treatment device using a magnetic field in accordance with an embodiment of the present invention.

Referring to FIG. 1, a treatment device 100 using a magnetic field (hereinafter, referred to as a magnetic field generating treatment device 100) in accordance with the present invention includes a magnetic field generating coil 110, a blower 120, a duct 130 guiding a fluid, a housing 140, and an internal electronic device 150.

The magnetic field generating coil 110 generates a pulse magnetic field through an applied current. The magnetic field generating coil 110 is disposed below a close contact surface 141 that closely contacts a portion of a body.

Although a circular coil disposed on a plane having a central axis perpendicular to the close contact surface 141 is provided as an example of the magnetic field generating coil 110 in this embodiment, the embodiment of the present invention is not limited thereto. For example, the magnetic field generating coil 110 may be various well-known types of coils that are applicable to the magnetic field generating treatment device.

The blower 120 supplies a fluid for cooling the magnetic field generating coil 110.

The blower 120 includes an inlet blower 120a supplying a fluid to the magnetic field generating coil 110 and an outlet blower 120b exhausting the fluid passed through the magnetic field generating coil 110 to the outside. The inlet blower 120a and the outlet blower 120b may be disposed at positions that are symmetric to each other with respect to the magnetic field generating coil 110.

The inlet blower 120a supplies a fluid from the outside to the inside of the magnetic field generating treatment device 100 in accordance with the present invention. Here, the inlet blower 120a may be one selected from a fan, a blower, a compressor, and a pump. Referring to FIG. 2, the inlet blower 120a is disposed below the magnetic field generating coil 110. The inlet blower 120a is disposed so that an angle θ between a fluid supply direction 121 from the inlet blower 120a and a central axis 111 of the magnetic field generating coil 110 is equal to or less than a right angle.

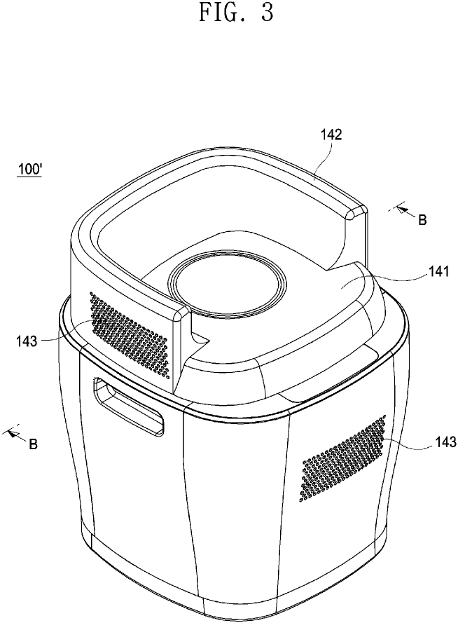

The outlet blower 120b exhausts a fluid flowing in the magnetic field generating treatment device 100. Here, the outlet blower 120b may be one selected from a fan, a blower, a compressor, and a pump. Referring to FIG. 3, the outlet blower 120b is disposed below the magnetic field generating coil 110. The outlet blower 120b is disposed so that an angle θ between a fluid supply direction 121 from the outlet blower 120b and a central axis 111 of the magnetic field generating coil 110 is equal to or less than a right angle.

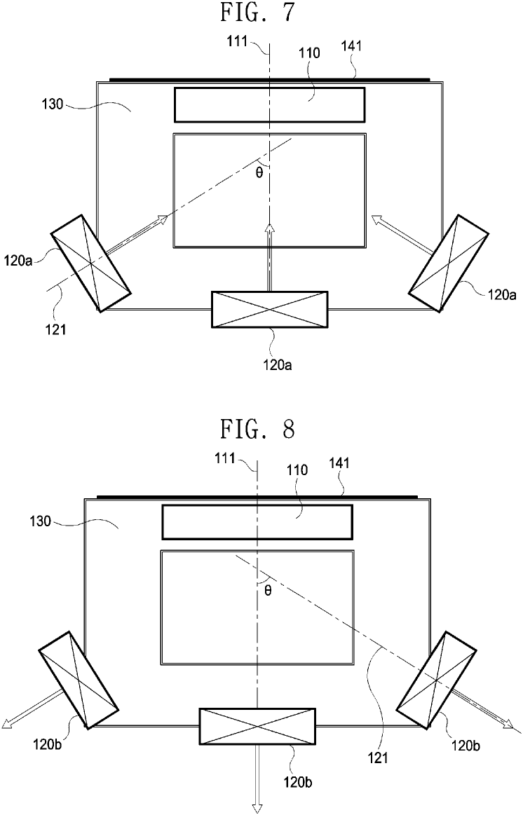

Here, the angle θ between the fluid supply direction 121 and the central axis 111 of the magnetic field generating coil 110 may be greater than 0° and less than 90°, preferably equal to or greater than 10° and equal to or less than 60°. This will be described later with reference to FIGS. 7 to 9.

The duct 130 guides a fluid between the blower 120 and the magnetic field generating coil 110.

Specifically, the duct 130 has one end 131a opened to the inlet blower 120a and the other end 131b opened to the outlet blower 120b. The duct 130 guides the fluid introduced from the inlet blower 120a to pass through the magnetic field generating coil 110 and be discharged through the outlet blower 120b.

The duct 130 is formed such that an internal cross-section of an area in which the magnetic field generating coil 110 is disposed is less than a cross-section of each of the one end 131a and the other end 131b. In other words, the duct 130 is formed such that a cross-section of an area in which a seated surface 132 is provided is less than that of each of the one end 131a and the other end 131b. Since a velocity of the fluid increases in an area in which the internal cross-section decreases, the duct 130 has a structure in which the velocity of the fluid increases in the area in which the magnetic field generating coil 110 is disposed.

The duct 130 includes the seated surface 132 on which the magnetic field generating coil 110 is seated at a position corresponding to the close contact surface 141.

The magnetic field generating coil 110 is seated on the seated surface 132, and the duct 130 includes a support 133 supporting the magnetic field generating coil 110 at a position corresponding to the seated position. The support 133 allows the magnetic field generating coil 110 to be spaced a predetermined distance from an inner circumferential surface of the duct 130. The fluid supplied from the inlet blower 120a flows through a space spaced by the support 133.

The support 133 may be rubber. The support 133 may serve to absorb a vibration generated in a process of operating the magnetic field generating coil 110.

The duct 130 includes a hole 135 defined in at least a portion of an area between the inlet blower 120a and the magnetic field generating coil 110.

The hole 135 allows a portion of the fluid introduced to the duct 130 to pass therethrough. The fluid passed through the hole 135 may cool the internal electronic device 150. As the duct 130 includes the hole 135, a portion of the fluid introduced from the inlet blower 120a may be used to cool heat generated from the magnetic field generating coil 110, and the rest of the fluid may be used to cool heat generated from the internal electronic device 150. Here, the number and size of the hole 135 may be determined so that about 70% of the fluid supplied to the hole 135 flows to the magnetic field generating coil 110, and about 30% of the fluid flows to the internal electronic device 150.

The duct 130 may simultaneously cool the magnetic field generating coil 110 and the internal electronic device 150 by including the hole 135. Through this, the magnetic field generating treatment device 100 in accordance with the present invention may increase a cooling efficiency through a compact structure instead of including a separate cooling unit for the internal electronic device 150.

The duct 130 may include a curved portion 136 obtained by bending at least a portion of the inner circumferential surface between the inlet blower 120a and the magnetic field generating coil 110.

Although the curved portion 136 is formed by bending a portion of the inner circumferential surface of the duct 130 in this embodiment, the embodiment of the present invention is not limited thereto. For example, the curved portion 136 may have an embossing shape or be formed in various methods such as a method of attaching a separate member to a portion of the inner circumferential surface of the duct 130.

The duct 130 may reduce a noise generated when a fan or a blower is used by including the curved portion 136. A noise generated when the fluid introduced from the inlet blower 120a contacts the curved portion 136 of the duct 130 may be less than that generated when the fluid introduced from the inlet blower 120a contacts other areas of the duct 130.

The housing 140 includes the close contact surface 141 supporting a body of a user. The close contact surface 141 closely contacts a portion of the body of the user, e.g., a hip of the user, when the user is seated. As illustrated, the close contact surface 141 may be formed as a flat surface or a surface having a predetermined curvature.

Also, the housing 140 may include a plurality of holes 143 for cooling. The holes 143 may be formed at a position corresponding to the blower 120.

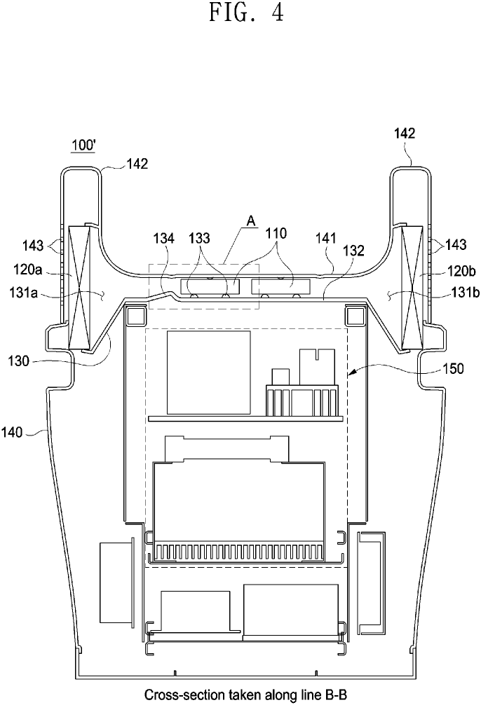

FIG. 3 is a perspective view illustrating a magnetic field generating treatment device in accordance with another embodiment of the present invention, and FIG. 4 is a cross-sectional view taken along line B-B and illustrating the magnetic field generating treatment device in accordance with another embodiment of the present invention.

A magnetic field generating treatment device 100′ in accordance with this embodiment has a similar overall configuration with the magnetic field generating treatment device 100 described with reference to FIGS. 1 and 2, but has a difference in components related to a protruding surface 142. Hereinafter, when the magnetic field generating treatment device 100′ in accordance with this embodiment is described, a detailed description on components common with those FIGS. 1 and 2 will be omitted, and only different components will be described.

The magnetic field generating treatment device 100′ in accordance with the present invention is used when the user is seated thereon.

The magnetic field generating treatment device 100′ in accordance with the present invention includes, as one component of a housing 140, a close contact surface 141 that closely contacts a portion of the body of the user, e.g., a hip of the user, and a protruding surface 142 that supports a portion of the body, e.g., a waist and a pelvis of the user. The magnetic field generating treatment device 100′ in accordance with the present invention suggests a chair-type treatment device that integrates a member for supporting the body when the user is seated, i.e., the protruding surface 142, so that the user is seated at an exact position even without coupling a separate member.

As illustrated in FIG. 3, the protruding surface 142 may have a chair backrest shape or a chair armrest shape. However, the embodiment of the present invention is not limited thereto. For example, the protruding surface 142 may have various shapes as long as the shape is capable of supporting the body of the user when the user is seated and guiding a seated position of the user.

The protruding surface 142 extends upward from the close contact surface 141 in at least a portion of an outer circumference of the close contact surface 141. The protruding surface 142 has a wall shape to have an inner accommodation space. In the accommodation space of the protruding surface 142, at least a portion of blowers 120a and 120b may be disposed.

The blowers 120a and 120b may be disposed on an extension line of an outer circumference of a magnetic field generating coil 110, and at least a portion of the blowers 120a and 120b may be disposed in the accommodation space of the protruding surface 142. The blowers 120a and 120b may be symmetrically disposed with respect to the magnetic field generating coil 110 in the accommodation space of the protruding surface 142.

A plurality of holes 143 for cooling may be defined in positions corresponding to the blowers 120. In accordance with this embodiment, the plurality of holes 143 for cooling may be defined in an outer surface of the protruding surface 142.

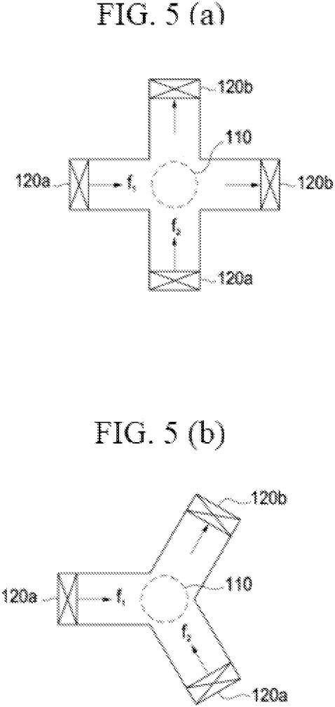

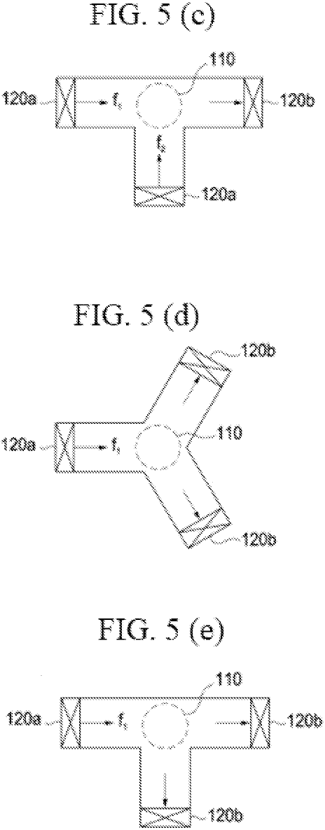

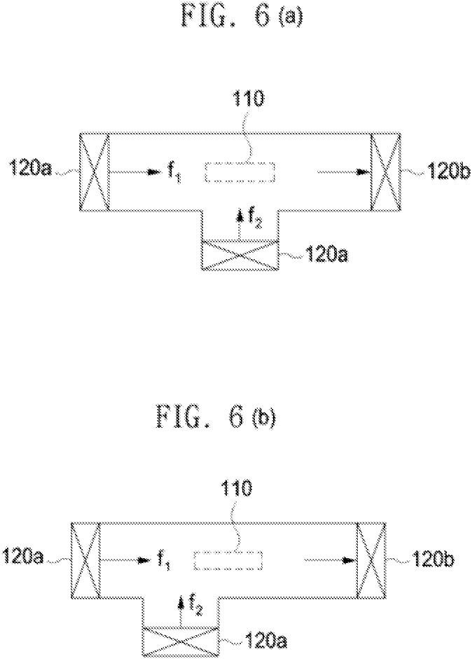

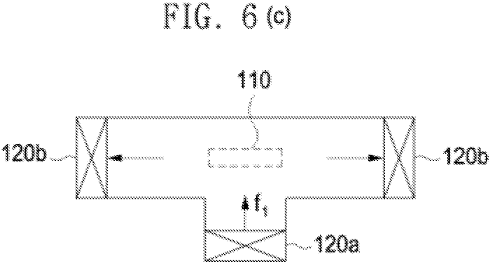

FIGS. 5 and 6 are schematic views for explaining various examples in which a plurality of blowers are disposed in magnetic field generating treatment devices in accordance with various embodiments of the present invention.

FIG. 5 illustrates, on a plane, a relationship of relative positions of the magnetic field generating coil 110 and the blowers 120a and 120b when the magnetic field generating treatment devices 100 and 100′ in accordance with various embodiments of the present invention are viewed from the top. This does not mean that the magnetic field generating coil 110 and the blowers 120a and 120b are disposed on the plane. The position relationship when the magnetic field generating treatment devices 100 and 100′ are viewed from the top is used to explain a cooling fluid flow caused by arrangement of the magnetic field generating coil 110 and the blowers 120a and 120b based on the magnetic field generating coil 110, excluding a distance difference between the close contact surface 141 and the blowers 120a and 120b or a stepped portion between the magnetic field generating coil 110 and the blowers 120a and 120b.

In other words, the blowers 120a and 120b in FIG. 5 may be disposed so that an angle θ between the fluid supply direction and the central axis 111 of the magnetic field generating coil 110 is equal to or less than a right angle as with the blowers 120a and 120b in FIG. 2 or disposed on an extension line of the outer circumference of the magnetic field generating coil 110 while at least a portion of the blowers 120a and 120b is disposed in the accommodation space of the protruding surface 142 as with the blowers 120a and 120b in FIG. 4.

Referring to FIG. 5, the blowers 120a and 120b may be provided in plurality to generate a cooling fluid flow having a plurality of paths passing through the magnetic field generating coil 110. Particularly, the inlet blower 120a may be provided in plurality to generate cooling fluid flows f1 and f2 having a plurality of paths passing through the magnetic field generating coil 110.

Each of the magnetic field generating treatment devices 100 and 100′ may include a plurality of blowers 120a each generating a first cooling fluid flow f1 and a second cooling fluid flow f2. That is, each of the magnetic field generating treatment devices 100 and 100′ may include a first blower 120a generating the first cooling fluid flow f1 in a direction parallel to a surface formed by the magnetic field generating coil 110 and a second blower 120a generating the second cooling fluid flow f2 in a direction different from the first cooling fluid flow f1 while passing through the magnetic field generating coil 110 in a direction parallel to the surface formed by the magnetic field generating coil 110.

Here, the magnetic field generating coil 110 is disposed on a path at which the first cooling fluid flow f1 from the first blower 120a overlaps the second cooling fluid flow f2 from the second blower 120a.

The first cooling fluid flow f1 and the second cooling fluid flow f2 may be arranged to form a right angle as illustrated in FIGS. 5a and 5e or arranged to form an obtuse angle as illustrated in FIG. 5b.

Each of the magnetic field generating treatment devices 100 and 100′ may further include an outlet blower 120b exhausting the fluid passed through the magnetic field generating coil 110. The outlet blower 120b may have a flow velocity and a flow rate, which are appropriately adjusted so that the first cooling fluid flow f1 and the second cooling fluid flow f2 generated from the inlet blower 120a cool the magnetic field generating coil 110 while passing through the magnetic field generating coil 110 instead of being returned to the inlet blower 120a again. The outlet blower 120b may be provided in plurality as illustrated in FIG. 5a, or the single outlet blower 120b may be provided as illustrated in FIGS. 5b and 5e.

As illustrated in FIG. 5a, each of the outlet blowers 120b and the inlet blowers 120a may be disposed symmetrically with respect to the magnetic field generating coil 110.

One outlet blower 120b may be provided to exhaust the fluid introduced from the plurality of outlet blowers 120a at once. Here, the outlet blower 120b may form a predetermined angle with each of the inlet blowers 120a as illustrated in FIG. 5b or may be disposed symmetrically with one inlet blower 120a with respect to the magnetic field generating coil 110 and form a right angle with another inlet blower 120a.

FIG. 6 illustrates, on a plane, a relationship of relative positions of the magnetic field generating coil 110 and the blowers 120a and 120b when the magnetic field generating treatment devices 100 and 100′ are viewed from one side. This does not mean that the magnetic field generating coil 110 and the blowers 120a and 120b are disposed on a straight line. The position relationship when the magnetic field generating treatment devices 100 and 100′ are viewed from one side is used to explain the cooling fluid flow caused by arrangement of the magnetic field generating coil 110 and the blowers 120a and 120b based on the magnetic field generating coil 110, excluding a distance difference between a side surface of the housing 140 and the blowers 120a and 120b or a stepped portion between the magnetic field generating coil 110 and the blowers 120a and 120b.

In other words, the blowers 120a and 120b in FIG. 6 may be disposed so that an angle θ between the fluid supply direction and the central axis 111 of the magnetic field generating coil 110 is equal to or less than a right angle as with the blowers 120a and 120b in FIG. 2 or disposed on an extension line of the outer circumference of the magnetic field generating coil 110 while at least a portion of the blowers 120a and 120b is disposed in the accommodation space of the protruding surface 142 as with the blowers 120a and 120b in FIG. 4.

Referring to FIG. 6, the blowers 120a and 120b may be provided in plurality to generate a cooling fluid flow having a plurality of paths passing through the magnetic field generating coil 110. Particularly, the inlet blower 120a may be provided in plurality to generate cooling fluid flows f1 and f2 having a plurality of paths passing through the magnetic field generating coil 110.

Each of the magnetic field generating treatment devices 100 and 100′ may include a plurality of blowers 120a each generating a first cooling fluid flow f1 and a second cooling fluid flow f2. That is, each of the magnetic field generating treatment devices 100 and 100′ may include a first blower 120a generating the first cooling fluid flow f1 in a direction parallel to a surface formed by the magnetic field generating coil 110 and a second blower 120b generating the second cooling fluid flow f2 in a direction perpendicular to the surface formed by the magnetic field generating coil 110.

Here, the magnetic field generating coil 110 is disposed on a path at which the first cooling fluid flow f1 from the first blower 120a overlaps the second cooling fluid flow f2 from the second blower 120a.

Each of the magnetic field generating treatment devices 100 and 100′ may further include an outlet blower 120b exhausting the fluid passed through the magnetic field generating coil 110. The outlet blower 120b may have a flow velocity and a flow rate, which are appropriately adjusted so that the first cooling fluid flow f1 and the second cooling fluid flow f2 generated from the inlet blower 120a cool the magnetic field generating coil 110 while passing through the magnetic field generating coil 110 instead of being returned to the inlet blower 120a again. As illustrated in FIGS. 6a and 6b, the single outlet blower 120b may be provided or a plurality of outlet blowers 120b may be provided.

As illustrated in FIGS. 6a and 6b, the first blower 120a generating the first cooling fluid flow f1 and the outlet blower 120b may be disposed symmetrically with respect to the magnetic field generating coil 110, and the second blower 120a generating the second cooling fluid flow f2 may be disposed below the magnetic field generating coil 110.

Here, the outlet blower 120b may be disposed directly below the magnetic field generating coil 110 to overlap the surface formed by the magnetic field generating coil 110 as illustrated in FIG. 6a or disposed below the magnetic field generating coil 110 in a state of being biased toward the first blower 120a based on the surface formed by the magnetic field generating coil 110. When the outlet blower 120b is disposed as in FIG. 6b, a cooling effect may improve because the fluid passes through the magnetic field generating coil 110 with a fast flow velocity as the first cooling fluid flow f1 and the second cooling fluid flow f2 overlap each other.

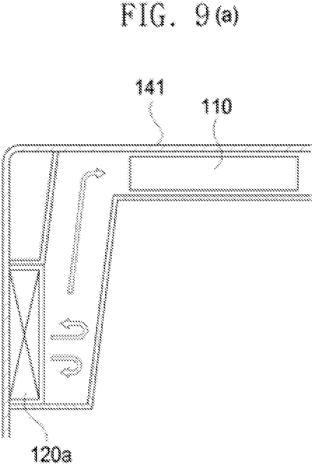

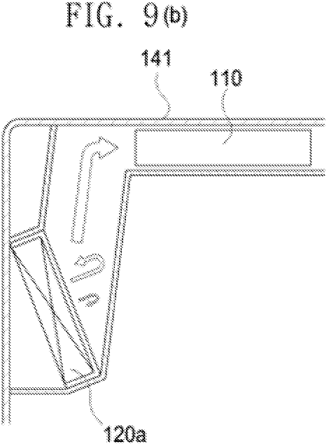

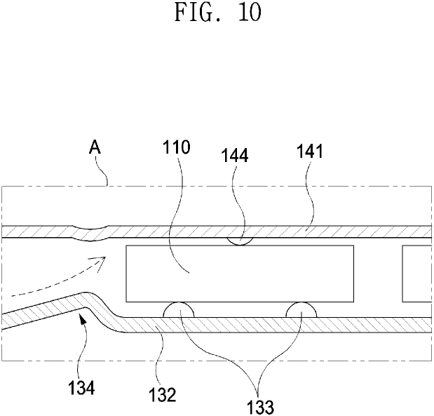

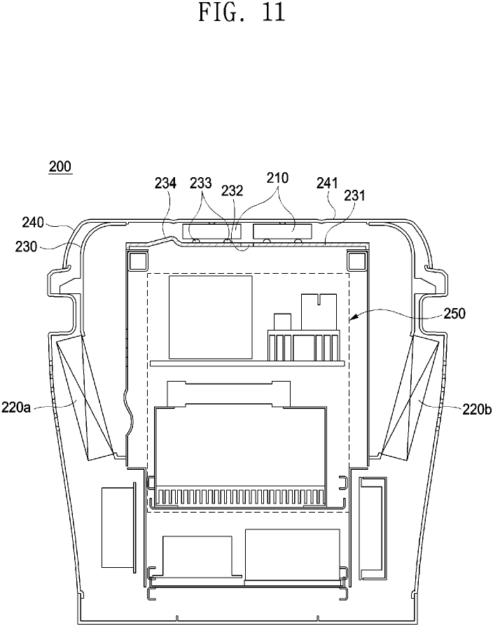

FIG. 7 is a schematic conceptual view illustrating a position of the inlet blower of the magnetic field generating treatment device in accordance with an embodiment of the present invention, FIG. 8 is a schematic conceptual view illustrating a position of the outlet blower of the magnetic field generating treatment device in accordance with an embodiment of the present invention, and FIG. 9 is a cross-sectional view for comparing and explaining a cooling structure in accordance with the present invention.

Referring to FIG. 7, the inlet blower 120a is disposed below the magnetic field generating coil 110. The inlet blower 120a is disposed so that an angle θ between the fluid supply direction 121 from the inlet blower 120a and the central axis 111 of the magnetic field generating coil 110 is equal to or less than a right angle. Referring to FIG. 8, the outlet blower 120b is disposed below the magnetic field generating coil 110. The outlet blower 120b is disposed so that an angle θ between the fluid supply direction 121 from the outlet blower 120b and the central axis 111 of the magnetic field generating coil 110 is equal to or less than a right angle.

Here, the angle θ between the fluid supply direction 121 and the central axis 111 of the magnetic field generating coil 110 may be greater than 0° and less than 90°, preferably equal to or greater than 10° and equal to or less than 60°.

Advantages of a case of forming a predetermined angle between the fluid supply direction 121 from the inlet blower 120a and the central axis 111 of the magnetic field generating coil 110 will be described with reference to FIGS. 9a and 9b.

As the angle θ between the fluid supply direction 121 from the inlet blower 120a and the outlet blower 120b and the central axis 111 of the magnetic field generating coil 110 is set to a preset angle, the magnetic field generating treatment device 100 may improve the cooling efficiency of the magnetic field generating coil 110 instead of arranging the blower 120 on the same plane as the magnetic field generating coil 110.

FIG. 9a is a view illustrating a case when the angle between the fluid supply direction 121 from the inlet blower 120a and the central axis 111 of the magnetic field generating coil 110 is 90°. In this case, the fluid supplied from the inlet blower 120a is not supplied toward the magnetic field generating coil 110 but returned to the inlet blower 120a by the inner circumferential surface of the duct 130. Since this arrangement does not effectively move the fluid supplied from the inlet blower 120a to the magnetic field generating coil 110, the cooling efficiency of the magnetic field generating treatment device 100 is degraded. However, FIG. 9b is a view illustrating a case when the fluid supply direction 121 from the inlet blower 120a forms a predetermined angle with the central axis 111 of the magnetic field generating coil 110. In this case, since the fluid supplied from the inlet blower 120a is directed toward the magnetic field generating coil 110, the fluid supplied from the inlet blower 120a may be effectively moved to the magnetic field generating coil 110.

Furthermore, as the angle θ between the fluid supply direction 121 from the inlet blower 120a and the outlet blower 120b and the central axis 111 of the magnetic field generating coil 110 is set to a preset angle, an overall volume of the magnetic field generating treatment device 100 in accordance with the present invention may be reduced. As illustrated in FIG. 1, as the groove is defined in an area of the housing 140 between the blower 120 and the magnetic field generating coil 110, the user may use the groove as a handle to thus improve user convenience. Also, as the angle θ between the fluid supply direction 121 from the inlet blower 120a and the outlet blower 120b and the central axis 111 of the magnetic field generating coil 110 is set to a preset angle, a limitation of introducing dusts existing at a bottom portion into the device may be prevented although one of a fan, a blower, a compressor, and a pump is used as the inlet blower 120a.

FIG. 10 is an enlarged cross-sectional view illustrating region A of FIGS. 2 and 4.

The duct 130 includes a seated surface 132 on which the magnetic field generating coil 110 is seated at a position corresponding to the close contact surface 141.

The magnetic field generating coil 110 is seated on the seated surface 132, and the duct 130 includes a support 133 supporting the magnetic field generating coil 110 at a position corresponding to the seated position. The support 133 allows the magnetic field generating coil 110 to be spaced a predetermined distance from the inner circumferential surface of the duct 130. The fluid supplied from the inlet blower 120a flows through a space spaced by the support 133.

The support 133 may be rubber. The support 133 may serve to absorb a vibration generated in a process of operating the magnetic field generating coil 110.

The duct 130 includes a protruding portion 134 protruding from the seated surface 132 toward the magnetic field generating coil 110. The protruding portion 134 is formed in at least a portion of the seated surface 132 formed between the inlet blower 120a and the magnetic field generating coil 110.

As illustrated in FIG. 10, the protruding portion 134 guides the fluid introduced from the inlet blower 120a to flow toward an upper portion of the magnetic field generating coil 110. In other wards, the protruding portion 134 guides the fluid introduced from the inlet blower 120a to flow toward an area between the magnetic field generating coil 110 and the close contact surface 141. The protruding portion 134 may improve the cooling efficiency of the portion closely contacting a portion of the body of the user by increasing a flow velocity and a flow rate of the fluid supplied to the area between the magnetic field generating coil 110 and the close contact surface 141.

Although the protruding portion 134 is formed by bending a portion of the inner circumferential surface of the duct 130 in this embodiment, the embodiment of the present invention is not limited thereto. For example, the protruding portion 134 may be formed in various methods such as a method of attaching a separate member to a portion of the inner circumferential surface of the duct 130.

The close contact surface 141 includes a support 144 protruding from a bottom surface of the close contact surface 141 to the magnetic field generating coil 110. Here, the close contact surface 141 may be a portion of the housing 140 or a portion of the duct 130.

The support 144 allows the magnetic field generating coil 110 to be spaced a predetermined distance from the close contact surface 141. The fluid supplied from the inlet blower 120a flows through a space spaced by the support 144 flows by the support 144. The support 144 may be rubber as with the support 133 formed on the seated surface 132. The support 144 may serve to absorb a vibration generated in a process of operating the magnetic field generating coil 110.

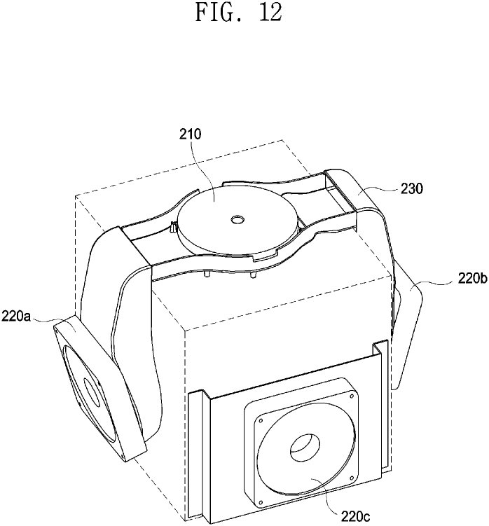

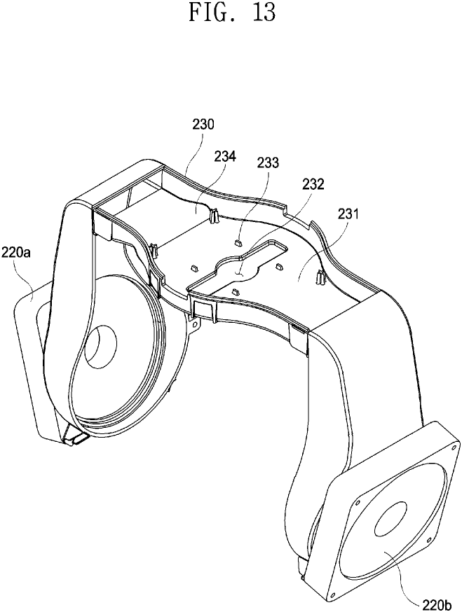

FIG. 11 is a cross-sectional view taken along line A-A and illustrating a magnetic field generating treatment device in accordance with another embodiment of the present invention, and FIG. 12 is a perspective view illustrating an example in which a blower contained in the magnetic field generating treatment device in accordance with the embodiment of FIG. 11 of the present invention.

A magnetic field generating treatment device 200 in accordance with this embodiment has an overall configuration similar to that of the above-described magnetic field generating treatment device 100, but has a difference in arrangement of a plurality of blowers 220a, 220b, and 220c and cooling fluid flows f1, f2, and f3 caused by the arrangement of the blowers.

The magnetic field generating treatment device 200 in accordance with the present invention includes a magnetic field generating coil 210, blowers 220a, 220b, and 220c, a duct 230 for guiding a fluid, a housing 240, and an internal electronic device 250. Hereinafter, a redundant description on a corresponding component will be omitted, and a configuration of the blowers 220a, 220b, and 220c and the duct 230 and the cooling fluid flows f1, f2, and f3 caused by the configuration will be described in detail.

Referring to FIGS. 11 and 12, the magnetic field generating treatment device 200 includes a first blower 220a, a second blower 220b, and a third blower 220c.

The first blower 220a and the second blower 220b may be disposed symmetrically with respect to the magnetic field generating coil 210. Each of the first blower 220a and the second blower 220b, as an inlet blower supplying a fluid to the magnetic field generating coil, may be one selected from a fan, a blower, a compressor, and a pump.

The first blower 220a and the second blower 220b may be disposed below the magnetic field generating coil, and the first blower 220a and the second blower 220b may be disposed so that an angle θ between a fluid supply direction from the first blower 220a and the second blower 220b and a central axis of the magnetic field generating coil 210 is equal to or less than a right angle as illustrated in FIGS. 7 and 8.

The magnetic field generating treatment device 200 in accordance with an embodiment of the present invention further includes a third blower 220c.

The third blower 220c discharges a fluid to the outside from the inside of the magnetic field generating treatment device 200. Particularly, the third blower 220c guides a fluid supplied from the first blower 220a and the second blower 220b to pass through the magnetic field generating coil 210 and the internal electronic device 250 and be discharged to the outside of the magnetic field generating treatment device 200.

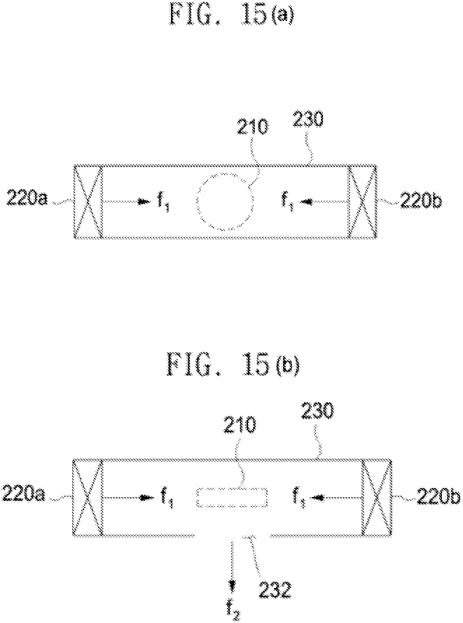

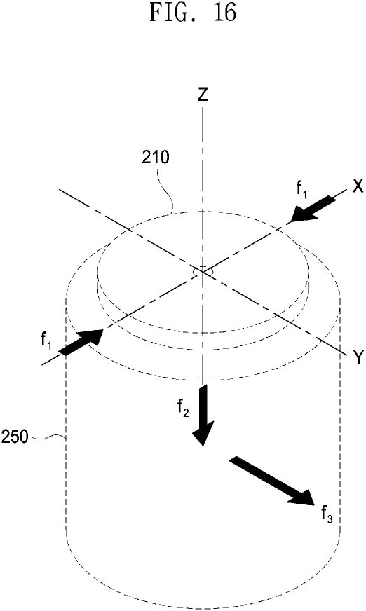

The cooling fluid flows f1, f2, and f3 caused by the first blower 220a, the second blower 220b, and the third blower 220c of the magnetic field generating treatment device 200 in accordance with the present invention will be described later with reference to FIGS. 15 to 17.

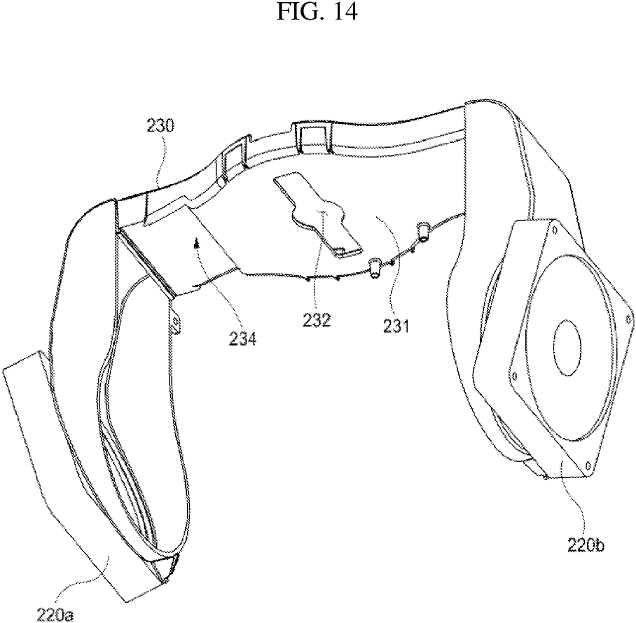

FIGS. 13 and 14 are perspective views illustrating a structure of a duct 230 contained in the magnetic field generating treatment device in accordance with the present invention.

The duct 230 guides a fluid between the blower 220a and 220b and the magnetic field generating coil 210.

Referring to FIGS. 13 and 14, the duct 230 has one end opened to the first blower 220a and the other end opened to the second blower 220b. The duct 230 guides the fluid supplied from the first blower 220a and the second blower 220b to flow toward the magnetic field generating coil 210.

The duct 230 may provide a seated surface 231 on which the magnetic field generating coil 210 is seated, and the seated surface 231 may be formed at a position corresponding to a close contact surface 241. The magnetic field generating coil 210 may be seated on the seated surface 231, and the duct 230 may include a support 233 supporting the magnetic field generating coil 210 at a position corresponding to the seated position.

The duct 230 includes an opening 232 formed by opening at least a portion of an area forming the seated surface 231. The opening 232 guides a fluid supplied from the first blower 220a and the second blower 220b to pass through the magnetic field generating coil 210 and be introduced toward the internal electronic device 250 disposed adjacent to the magnetic field generating coil 210.

As illustrated in FIG. 111, the internal electronic device 250 may be disposed below the magnetic field generating coil 210, and the opening 232 may be defined in a region between the magnetic field generating coil 210 and the internal electronic device 250.

The duct 230 may include a protruding portion 234 protruding from the seated surface 231 toward the magnetic field generating coil 210. As described in the magnetic field generating treatment device 100, the protruding portion 234 may be formed in at least a portion of the seated surface 132 formed between the first blower 220a or the second blower 220b and the magnetic field generating coil 210 and guide the fluid introduced from the inlet blowers 220a and 220b to flow toward an upper portion of the magnetic field generating coil 210.

FIGS. 15 to 17 are schematic views for explaining a cooling fluid flow in the magnetic field generating treatment device in accordance with the embodiment of FIG. 11.

Specifically, FIGS. 15A and 15B illustrate, on a plane, a relationship of relative positions of the magnetic field generating coil 210 and the blowers 220a and 220b when the magnetic field generating treatment devices 200 is viewed from the top and the side.

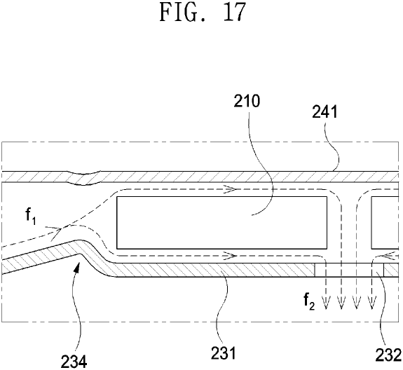

FIG. 16 is a three-dimensional view illustrating cooling fluid flows f1, f2, and f3 generated by a plurality of blowers 220a, 220b, and 220c based on the magnetic field generating coil 210, and FIG. 17 is a view illustrating the cooling fluid flows f1, f2, and f3 in a region adjacent to the magnetic field generating coil 210.

As described above in FIGS. 5 and 6, although the magnetic field generating coil 210 and the blowers 220a and 220b are disposed on the same plane in FIGS. 15A and 15B, the embodiment of the present invention is not limited thereto. That is, the cooling fluid flows f1 and f2 caused by arrangement of the magnetic field generating coil 210 and the blowers 220a and 220b is described based on the magnetic field generating coil 210, excluding a distance difference between the close contact surface 141 and the blowers 220a and 220b or a stepped portion between the magnetic field generating coil 210 and the blowers 220a and 220b.

Referring to FIGS. 15 to 17, a first blower 220a and a second blower 220b may generate the cooling fluid flow f1 having a plurality of paths passing through the magnetic field generating coil 210. Each of the inlet cooling fluid flows f1 may cool a plurality of side surfaces of the magnetic field generating coil 210 while passing through the magnetic field generating coil 210 in a direction parallel to a surface formed by the magnetic field generating coil 210. For example, the first blower 220a and the second blower 220b may be disposed symmetrically with respect to the magnetic field generating coil 210 and each generate the cooling fluid flow f1 introduced to the magnetic field generating coil 210.

Here, the cooling fluid flow f1 serves to generate a pulse magnetic field and cool heat generated from the magnetic field generating coil 210. The cooling of the magnetic field generating coil 210 may be more intensively and uniformly performed through the cooling fluid flow f1 having the plurality of paths generated by the first blower 220a and the second blower 220b.

The opening 232 defined in the duct 230 generates the cooling fluid flow f2 so that the cooling fluid flow f1 supplied from the first blower 220a and the second blower 220b to pass through the magnetic field generating coil 210 and be introduced toward the internal electronic device 250 disposed adjacent to the magnetic field generating coil 210. Here, the cooling fluid flow f2 may cool heat generated in a process of operating the internal electronic device 250.

Although not shown, the duct 230 may include a hole that allows a portion of a fluid introduced to at least a portion of a region between the first blower 220a or the second blower 220b and the magnetic field generating coil 210 to pass therethrough, and at least a portion of the fluid supplied from the inlet blower may be introduced to the internal electronic device 250 from the hole. Here, the hole guides the fluid to be introduced toward the internal electronic device 250 before at least a portion of the fluid supplied from the inlet blower reaches the magnetic field generating coil 210. The cooling fluid flow f2 may cool heat of the internal electronic device 250 of the magnetic field generating treatment device 200 in accordance with the present invention together with the fluid introduced from the hole.

The duct 230 may achieve an effect of simultaneously cooling the magnetic field generating coil 210 and the internal electronic device 250 by including the opening 232 and the selective hole. Through this, the magnetic field generating treatment device 200 in accordance with the present invention may increase a cooling efficiency with a compact structure excluding a separate cooling unit for the internal electronic device 250.

Thereafter, the third blower 220c generate the cooling fluid flow f3 so that the fluid supplied from the first blower 220a and the second blower 220b to pass through the magnetic field generating coil 210 and the internal electronic device 250 and be discharged to the outside of the magnetic field generating treatment device 200.

The cooling fluid flows f1, f2, and f3 may absorb the heat generated from the magnetic field generating coil 210 and the internal electronic device 250 to increase a temperature of the fluid while the fluid is introduced into and discharged from the magnetic field generating treatment device 200.

The cooling fluid flow f1 supplied from the first blower 220a and the second blower 220b and the cooling fluid flow f2 guided by the opening 232 may have approximately perpendicular relative flows as illustrated in FIGS. 15 to 17. Also, the cooling fluid flow f2 introduced to the internal electronic device 250 through the opening 232 and the cooling fluid flow f3 discharged to the outside of the magnetic field generating treatment device 200 by the third blower 220c may have approximately perpendicular relative flows as illustrated in FIG. 16. However, this embodiment shows the fluid flows and does not represent that the fluid flows are perpendicular to each other, and the scope of the present invention is not limited thereto.

Each of the magnetic field generating treatment devices 100, 100′, and 200 may include components greater or less than the above-described components. Also, each of the components may be constituted as a separate chip, module, or device, or contained in one device.

The description of the present invention is intended to be illustrative, and those with ordinary skill in the technical field of the present invention will be understood that the present invention can be carried out in other specific forms without changing the technical idea or essential features. Thus, the above-disclosed embodiments are to be considered illustrative and not restrictive.

1. A treatment device using a magnetic field, comprising:
a magnetic field generating coil disposed below a close contact surface that closely contacts a portion of a body;
a first blower and a second blower disposed symmetrically with respect to the magnetic field generating coil to supply a fluid toward the magnetic field generating coil; and
a duct configured to provide a seated surface on which the magnetic field generating coil is seated and guide the fluid supplied from the first blower and the second blower toward the magnetic field generating coil,
wherein the duct comprises an opening formed as at least a portion of an area forming the seated surface is opened.

2. The treatment device of claim 1, further comprising an internal electronic device disposed adjacent to the magnetic field generating coil,
wherein the opening guides the fluid so that the fluid supplied from the first blower and the second blower passes through the magnetic field generating coil and then is introduced toward the internal electronic device.

3. The treatment device of claim 2, further comprising a third blower configured to discharge the fluid to the outside from the inside of the treatment device using a magnetic field,
wherein the third blower guides the fluid so that the fluid supplied from the first blower and the second blower is discharged to the outside of the treatment device using a magnetic field through the magnetic field generating coil and the internal electronic device.

4. The treatment device of claim 2, wherein the internal electronic device is disposed below the magnetic field generating coil,
the opening is defined in a region between the magnetic field generating coil and the internal electronic device, and
the duct guides the fluid so that the fluid supplied from the first blower and the second blower passes through the magnetic field generating coil in a direction parallel to a surface formed by the magnetic field generating coil, and the fluid passed through the magnetic field generating coil is supplied to the internal electronic device in a direction perpendicular to the surface formed by the magnetic field generating coil.

5. The treatment device of claim 1, wherein the duct comprises a hole configured to allow a portion of the fluid introduced to at least a portion of a region between the first blower or the second blower and the magnetic field generating coil to pass therethrough, and
the hole guides the fluid so that at least a portion of the fluid supplied from the first blower or the second blower is introduced toward the internal electronic device before being reached to the magnetic field generating coil.
